(19) United States
(12) Patent Application Publication
(10) Pub. No.: US 2025/0123294 A1
(43) Pub. Date: Apr. 17, 2025

(54) METHODS AND DEVICES FOR DETECTION OF ORTHOGONAL CLASSES OF DISEASE BIOMARKERS

(71) Applicant: INNOTECH PRECISION MEDICINE, INC., Boston, MA (US)

(72) Inventor: Roya Khosravi-Far, Boston, MA (US)

(21) Appl. No.: 18/652,397

(22) Filed: May 1, 2024

Related U.S. Application Data
(60) Provisional application No. 63/463,204, filed on May 1, 2023.

(51) Int. Cl. G01N 33/68; G01N 33/543

(52) U.S. Cl. CPC G01N 33/6893; G01N 33/54333; G01N 2333/4704; G01N 2333/71

(57) ABSTRACT

In one aspect, a method for screening an individual for head and neck cancer, which comprising: acquiring a saliva sample from the individual, analyzing the saliva sample to detect one or more of HPVs and at least one of a phenotypic biomarker, a regulatory biomarker, a microbiome-derived biomarker, and a metabolomic biomarker dysregulated due to HPV infection, wherein detection of both of the HPV and at least one of said biomarkers indicates the individual is at risk of head and neck cancer.

### RELATED APPLICATION

The present application claims priority to provisional application No. 63/463,204 field on May 1, 2023, and the contents of which are herein incorporated by reference in their entirety.

### SEQUENCE LISTING

The instant application contains a Sequence Listing which has been submitted electronically in XML format and is hereby incorporated by reference in its entirety. Said XML copy, created on Dec. 31, 2024, is named 119275-0032-004-101_SL.xml and is 22,408 bytes in size.

### TECHNICAL FIELD

The present teachings are directed to diagnostic methods and systems for detection of orthogonal classes of disease biomarkers.

### BACKGROUND

The diagnosis and early detection of a variety of diseases can be crucial in obtaining positive treatment results. Accurate diagnosis of diseases at point-of-care will allow timely and effective treatment and will save lives. For example, cancer is a multi-step process that takes many years (in some cases up to 15 years) to develop. The therapeutic outcomes of cancer treatments can be dramatically improved if cancer is detected at an early stage.

Accordingly, there is a need for improved diagnostic methods and systems for accurate and preferably early detection of a variety of diseases.

### SUMMARY

In one aspect, a method for screening an individual for head and neck cancer is disclosed, which comprises: acquiring a saliva sample from the individual, analyzing the saliva sample to detect one or more of Human Papilloma Viruses (HPV) and at least one of a phenotypic biomarker, a regulatory biomarker, a microbiome-derived biomarker, and a metabolomic biomarker dysregulated due to HPV infection, wherein detection of both of the HPV and at least one of said biomarkers indicates the individual is at risk of head and neck cancer.

By way of example, the phenotypic biomarker can be a biomarker that is associated with a cellular process affected (dysregulated) by the HPV infection. For example, the phenotypic biomarker can be any of a protein, an mRNA or a miRNA and the regulatory biomarker can be an miRNA.

The step of analyzing the saliva sample to detect HPV can include detecting at least one nucleotide target sequence associated with the HPV genome. In addition, or alternatively, the HPV can be detected via detection of an mRNA generated via transcription of the HPV genome in an infected host cell.

In various embodiments, the at least one protein biomarker can be p-16 protein and/or EGF (epidermal growth factor) receptor.

By way of example, the detection of HPV (e.g., via, the detection of its nucleic acid sequences or transcripts) together with p-16 and/or EGF protein or mRNA that are increased in response to HPV expression in a host cell) in a biological sample (e.g., a saliva sample) acquired from an individual can indicate that the individual is at risk of an HPV-derived cancer, e.g., head-and-neck cancer.

In various embodiments, once it is determined that an individual is at the risk of having head-and-neck cancer, the individual may be recommended for confirmatory follow-up tests, such as MRI imaging, etc.

In a related aspect, an assay for detecting a target protein in a biological sample is disclosed, which includes incubating, in presence of a substrate, the sample with a first antibody exhibiting specific binding to a first epitope of a target protein, and a second antibody exhibiting specific binding to a second epitope of the target protein to form an incubating mixture of the sample and the antibodies, wherein the first antibody is further configured for coupling to the substrate and the second antibody is conjugated to a nucleotide sequence. By way of example, the duration of the incubation period can be in a range of about 30 minutes to about 40 minutes. Subsequently, a wash step of the incubating mixture is performed to remove components thereof that are not bound to the substrate. This is followed by using an RPA-Exo assay to amplify and detect the oligonucleotide sequence, thereby detecting the target protein.

In various embodiments, the substrate can be functionalized with Streptavidin and the first antibody can be biotinylated for binding to the Streptavidin. In some such embodiment, the substrate can correspond to the surface of a plurality of magnetic beads, where the magnetic beads can be collected via application of a magnetic field to the incubation mixture. After the collection of the magnetic beads, a wash step can be performed followed by an RPA-Exo assay to detect the target protein.

In a related aspect, a method for screening an individual for a cancer mediated by HPV infection is disclosed, which includes acquiring a biological specimen from the individual, and analyzing the biological specimen to detect HPV and at least one a phenotypic biomarker, a regulatory biomarker, a metabolomic biomarker, and a microbiome-derived biomarker dysregulated via the HPV infection, where the detection of both the HPV and at least one of the biomarkers indicates the individual is at risk of the HPV-mediated cancer.

By way of example, the HPV-mediated cancer can be any of a cervical cancer, a head-and-neck cancer, a penile cancer, a vaginal caner, a valvar cancer and an anal cancer.

In various embodiments, the phenotypic biomarker can be associated with a cellular molecular process affected (dysregulated) by the HPV infection. The phenotypic biomarker can be any of a protein and an mRNA and the regulatory biomarker can be an miRNA. By way of example, the phenotypic biomarker can be p-16 protein and/or EFG receptor.

In various embodiments, the biological specimen can be analyzed to detect at least one target nucleotide sequence associated with the HPV genome for the detection of HPV in the specimen. In addition, or alternatively, the HPV can be detected in the biological specimen via the detection of an mRNA associated with the transcription of the HPV genome in an infected host cell.

In various embodiments, once an individual is identified as being at risk of having an HPV-derived cancer, the individual is recommended to obtain follow-up tests, such as MRI imaging.

In a related aspect, a method for detecting a disease is disclosed, which includes acquiring a biological sample from an individual, analyzing the biological sample to detect at least two orthogonal disease biomarkers, when present in the biological sample, wherein detection of the two orthogonal disease biomarkers indicates that the individual is at risk of having the disease.

In various embodiments, one of the at least two orthogonal disease biomarkers include at least a target protein and another one of the at least two orthogonal disease biomarkers includes a target nucleotide sequence.

In various embodiments, any of the target protein and the target nucleotide sequences is indicative of presence of a pathogen in the biological sample. By way of example, the pathogen can be a virus.

The above method can be employed for the detection (diagnosis) of a variety of different diseases. Some examples of such diseases include, without limitation, a cancer, a neurodegenerative disease, a hematological disease, an inflammatory disease, and an infectious disease.

In a related aspect, a system for detection of at least two classes of disease biomarkers, in a biological specimen is disclosed, which includes a cartridge frame having a sample well for receiving a biological specimen, a first lysis chamber in fluid communication with the sample well for receiving a first portion of the biological specimen and preparing the first sample portion for detection of at least one target protein, and a second lysis chamber in fluid communication with the sample well for receiving a second portion of the sample and preparing the second sample portion for detection of at least one target nucleic acid sequence. The system further includes a wash buffer blister pack containing a wash buffer and a collection well in fluid communication with the wash buffer blister pack. In addition, the system includes at least one protein detection well in fluid communication with the first lysis chamber and with the collection well and configured for detection of at least one target protein as well as at least one nucleic acid detection well in fluid communication with said second lysis chamber for receiving the second specimen portion and configured for detection of at least one target nucleic acid sequence. In various embodiments, the first lysis chamber contains at least one lysing agent for preparing a specimen for protein detection and the second lysis chamber contains at least one lysing agent for preparing a specimen for nucleic acid detection. Some examples of suitable lysing reagents are disclosed in the afore-mentioned '393 patent.

In various embodiments, the at least one protein detection well contains a plurality of magnetic particles functionalized with Streptavidin, at least a first antibody exhibiting specific binding to a first epitope of the target protein and at least a second antibody exhibiting binding to a second epitope of the target protein, where the first antibody is biotinylated for binding the Streptavidin so as to couple to the magnetic particles, and the second antibody includes at least one oligonucleotide sequence.

The cartridge can also include a first valve for regulating fluid communication between the first lysis chamber and said at least one protein detection well and a second valve for regulating fluid communication between the second lysis chamber and the at least one nucleic acid detection well. The valves can be operated under the control of a controller.

By way of example, the biological specimen can include any of a biofluid, a liquid biopsy, and a tissue-biopsy derived specimen.

The system can be employed for detecting (diagnosing) a variety of diseases, such as cancer, a neurodegenerative disease, a hematological disease, an inflammatory disease. By way of example, the disease can be any of cervical cancer, a head-and-neck cancer, penile cancer, and valvar cancer.

In a related aspect, a system for detection of at least two classes of disease biomarkers for diseases, in a biological specimen, is disclosed, which includes a cartridge having a sample port configured to receive the biological specimen, a first reservoir containing a first buffer for preparing a sample of the biological specimen for detection of a first class of the cancer biomarkers, a second reservoir containing a second buffer for preparing the sample for detection of a second class of the cancer biomarkers, a first sensor configured to detect the first class of the cancer biomarkers in a portion of the sample processed via the first buffer, and a second sensor configured to detect the second class of the cancer biomarkers in a portion of the sample processed via the second buffer.

At least one of said first and said second buffer comprises a lysing agent for processing the specimen.

Similar to the previous embodiments, the biological specimen comprises any of a biofluid, a liquid biopsy, and a tissue-biopsy derived specimen. By way of example, the system can be used for detection/diagnosis of cancer, a neurodegenerative disease, a hematological disease, an inflammatory disease. By way of example, the disease can be any of cervical cancer, a head-and-neck cancer, penile cancer, and valvar cancer.

Further understanding of various aspects of the present teachings can be achieved by reference to the following detailed description in conjunction with the associated drawings, which are described briefly below.

### BRIEF DESCRIPTION OF THE DRAWINGS

FIG. 1A is a flow chart depicting various steps in an embodiment of the present teachings for detecting a disease by analyzing a biological sample obtained from an individual,

FIG. 1B is a flow chart depicting various steps in an embodiment of the present teachings for detecting an HPV-mediated cancer,

FIG. 2 is a diagram comparing a conventional workflow for the detection of cervical cancer with a workflow according to various embodiments of the present teachings,

FIG. 3A is a flow chart depicting various steps performed in an embodiment of an assay according to the present teachings for detecting a target protein in a biological sample,

FIG. 3B is a diagram depicting various reactants utilized in the assay described in connection with FIG. 3A and their respective interactions,

FIG. 4 depicts a prior art cartridge that can be modified based on the present teachings to perform various embodiments of methods according to the present teachings,

FIG. 5 is a top view of a device to be utilized to perform multiplexed detection of target proteins and nucleotide sequences,

FIG. 6 is a schematic view of a cartridge in accordance with an embodiment of the present teachings, and

FIG. 7 is a schematic diagram depicting an example of a controller/fluorescence reader that can be used to control a cartridge according to various embodiments and detect fluorescent radiation generated in various wells of the cartridge.

### DETAILED DESCRIPTION

The present teachings are related to the detection of orthogonal disease biomarkers in complex diseases such as cancer, neurodegenerative diseases, hematological diseases, among others. As discussed in more detail below, in various embodiments, the detection of orthogonal disease biomarkers is achieved via introduction of a sample (herein also referred to as a specimen) into a cartridge that is configured to detect two or more orthogonal disease biomarkers. By way of example, in various embodiments, different sample-processing may be employed to prepare a specimen for the detection of different biomarkers therein. However, in many embodiments, the same detection modality is employed for the detection of different biomarkers, e.g., fluorescent detection. Further, in various embodiments, the preparation and detection of multiple biomarkers in a single specimen is carried out on the same cartridge.

For example, as discussed in more detail below, some embodiments provide a cartridge that includes at least one sensor for detection of one or more genetic components of an HPV virus, such as the detection of high-risk HPVs such as HPV 16, HPV 18, HPV 31, HPV 11 nucleic acids, including DNA, and mRNA and at least another sensor for the detection of genes, proteins, mRNA, miRNA, microbiome, metabolomic or other biomarkers associated with an HPV mediated cancer, such as cervical cancer or head and neck cancer or host response biomarkers. Further, in some embodiments, a cartridge according to the present teachings can also provide multiplexed detection of one or more genome nucleotide sequences, phenotypic biomarkers (e.g., protein, mRNA, miRNA), as well as one or more target metabolites, and microbiome-derived biomarkers, among other factors, such as those discussed below.

In various embodiments, a single specimen acquired from an individual is used for the detection of HPV as well as one or more biomarkers that together with the HPV detection would indicate that the individual is at risk of an HPV-mediated cancer, such as head-and-neck cancer. For example, a portion of a single specimen is utilized for the detection of HPV and another portion of the specimen is used for the detection of the biomarkers. In some embodiments, rather than splitting the specimen into different portions, the specimen is introduced into a single well and a multiplexed detection of HPV (e.g., via the detection of a genomic sequence or mRNA associated with the transcription of the HPV in a host cell) and one or more of the biomarkers discussed herein is performed. In many embodiments, the same detection modality is employed for the detection of HPV and one or more biomarkers in a single specimen (e.g., saliva) obtained from the individual. In addition, in various embodiments, the same statistical analysis algorithms are employed for analysis of signals, e.g., fluorescence signals, associated with the detection of HPV and the biomarkers. The detection of not only HPV but also an associated biomarker from a single specimen can advantageously increase the accuracy of screening for an HPV-derived cancer (such as the head-and-neck cancer). In addition, the use of the same statistical analysis in various embodiments, can increase the screening accuracy.

More generally, the detection of multiple orthogonal disease biomarkers from the same specimen using the same detection modalities, and optionally the same statistical analysis method, can improve the accuracy of the determination that an individual is at the risk of having the disease.

The phrase “orthogonal disease biomarkers” as used herein refers to biomarkers that can provide complementary information about a disease. By way of example, such orthogonal biomarkers can belong to different classes of biomarkers, such as, genetic, metabolic, phenotypic, microbiome-derived, and epigenetic biomarkers.

The term “biomarker” refers to a biological molecule found in biological specimens such as body fluids, or tissues that can provide a sign of a normal or abnormal process, or of a condition or disease.

The term “genetic biomarker,” as used herein, refers to a genomic nucleotide sequence that can indicate, for example, the presence of a pathogen (e.g., a virus) in a biological specimen.

The term “phenotypic biomarker,” as used herein, refers to any of a protein, a regulatory, transcription, or metabolic factor (compound) whose normal concentration can be modulated (dysregulated) due to the onset and/or progression of a disease. Some examples include a protein, an mRNA, or an miRNA.

The term “metabolomic biomarker,” as used herein, refers to biomarkers that are the small molecule substrates, intermediates, and products of cell metabolism. Metabolomic biomarkers provides a direct “functional readout of the physiological state” of an organism.

The term “microbiome-derived biomarker,” as used herein, refers to microbial signatures derived from the composition, diversity, and activity of microorganisms inhabiting a particular environment or organism, particularly the human body. These biomarkers can be based on various aspects of the microbiome, including the types and abundance of bacteria, archaea, viruses, fungi, and other microorganisms present in each sample.

The term “epigenetic biomarker,” as used herein, refers to molecular indicators that reflect changes in gene expression or regulation without alterations to the underlying DNA sequence. They encompass various epigenetic modifications such as DNA methylation, histone modifications, and non-coding RNAs.

Accurate diagnosis of diseases at point-of-care will allow timely and effective treatment and will save lives. Over the last 4 decades, the investigation into the hallmarks of cancer has demonstrated a complex of genotypic, phenotypic, metabolic reprogramming, non-mutational epigenetics, polymorphic microbiomes, host-response/immune response, and disrupted cellular matrix components among other characteristics (Reviewed in “Hallmarks of Cancer: New Dimensions,” by Hanahan, published in Cancer Discovery, vol. 12, Issue 1, 2022, which is herein incorporated by reference in its entirety). Thus, a simplistic evaluation of just one class of disease biomarkers cannot provide early and accurate diagnosis of cancer or for that matter other complex diseases such as neurodegenerative diseases, etc.

Cancer is a multi-step process that takes many years (in some cases up to 15 years) to develop. Multi-modal detection of two or more of genotypic, epigenetic, phenotypic, immune response, gene mutations, single nucleotide polymorphisms, metabolomic, DNA methylation, mRNA, miRNA, long noncoding RNAs (lncRNA), competing endogenous RNAs (ceRNAs), Circular RNA (circ-RNA) biomarkers simultaneously from the same specimen will allow early and timely detection of cancer at a stage of the disease that is curable. This will reduce unnecessary procedures that many patients are forced to undergo.

As an example, breast cancer is a heterogeneous disease at molecular level. Some molecular features include activation of human epidermal growth factor receptor 2 (HER2), activation of hormone receptors (estrogen and progesterone receptor) and/or BRCA mutations. While the presence of a germline BRCA mutation in a subject can indicate their predisposition to breast cancer and ovarian, pancreatic, and other cancers, BRCA mutation alone does not demonstrate that the subject at the time of testing has any of these cancers. Therefore, a rapid, multi-omics, and accurate point of care testing for screening and monitoring of patients with BRCA mutations can prevent unnecessary mastectomy, oophorectomy, or other procedures without any evidence of cancer.

More recently additional biomarkers have been discovered that may be important for early detection of breast cancer. By way of example a hsa_circRNA_002082 and hsa_circRNA_400031 may function as ceRNAs to serve key roles in breast cancer epithelial-mesenchymal transition (See, e.g., “Identification of epithelial-mesenchymal transition-related circRNA-miRNA-ceRNA regulatory network in breast cancer,” by Sang et al., published in Pathol Res Pract. 2022 September, 216 (9), which is herein incorporated by reference in its entirety). Additionally, promoter hypermethylation has been linked to the inactivation of tumor suppressor genes and is an early event in carcinogenesis. Indeed, hypermethylated tumor suppressor genes are frequently found in patient-derived BC tissues and peripheral blood biospecimens (98. doi: 10.3892/ijo.2021.5278).

More than 95% of cervical cancer cases are due to the human papillomavirus (HPV). The WHO has updated their guidelines for detection of cervical cancer and encouraged HPV tests as the primary test for cervical screening. However, the presence of HPVs (high risk HPV 16, HPV 18, HPV 31, HPV 35, and others) does not demonstrate that the patient has cervical cancer as many HPV infections can clear without causing cervical cancer. While a rapid and accurate point-of-care, point-of-need molecular test for the detection of HPV nucleic acid can significantly improve cervical cancer testing, it results in a high rate of false positives. There are those who are positive for HPV but do not have cervical cancer and their HPV can be cleared without causing cervical cancer. As a result, many individuals who are positive for HPV can be subjected to unnecessary and invasive procedures such as colposcopy as well as enduring emotional distress. Combining detection of HPV with other orthogonal biomarkers of cervical cancer will reduce these false results. In the U.S.A., HPV positive tests are flexed for cytology. However, in many instances, including in disadvantaged populations, follow up of a positive test can become challenging and some patients with cervical cancer are not receiving timely treatment.

Therefore, for diagnosis of complex diseases, an innovative diagnostic platform is needed that detects multiple classes of disease biomarkers in a multiplexed fashion from the same specimen and for accurate and early disease diagnosis and at patient's side. As discussed in more detail below, in various embodiments, such a platform is provided that employs a cartridge that allows detection of multiple classes of disease biomarkers from the same specimen.

Methylation levels of several DNA biomarkers, i.e., ASCL1/LHX8 have also been shown to significantly increase with increasing severity of cervical cancer. These markers can be detected in urine. In one study, analysis of these markers in patient specimens have resulted in an AUC of 0.84 for CIN3+ detection in urine, corresponding to an 86% sensitivity at a 70% specificity (See, “HPV and DNA methylation testing in urine for cervical intraepithelial neoplasia and cervical cancer detection,” van der Held et al., published in Clin. Cancer Res 2022 May 13: 28 (10): 2061-2068).

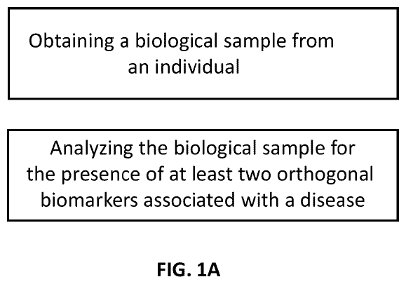

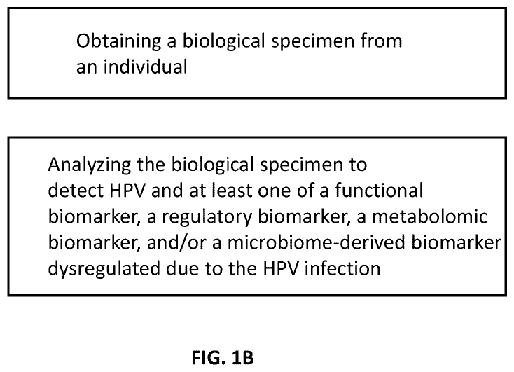

With reference to the flow chart of FIG. 1A, a method for detection of a disease according to an embodiment includes obtaining a biological specimen from an individual and analyzing the biological specimen for presence of at least two orthogonal biomarkers associated with the disease, where the detection of the at least two orthogonal biomarkers indicates that the individual is at the risk of having the disease. By way of example, the orthogonal biomarkers can include any of genetic biomarkers, phenotypic biomarkers, metabolomic biomarkers and/or microbiome-derived biomarkers. In various embodiments, the method for the detection of a disease according to the present teachings can be performed at the point-of-care.

HPV mediated cancers, such as cervical cancer and head-and-neck cancer constitute a class of diseases for which the detection methods according to various embodiments are suitable. By way of example and with reference to the flow chart of FIG. 1B, in a method according to various embodiments for detecting an HPV-mediated cancer, a biological specimen is acquired from an individual. The biological specimen is analyzed to detect HPV and at least one of a phenotypic biomarker, a regulatory biomarker, a metabolomic biomarker, and/or a microbiome-derived dysregulated due to the HPV infection. The detection of both the HPV and at least one of the biomarkers indicates that the individual is at the risk of having the HPV-mediated cancer.

By way of example, such a method can be employed for the detection of any of cervical cancer, head-and-neck cancer, vaginal and vulvar cancers, and anal cancer. In various embodiment, the methods according to the present teachings can be utilized for point-of-care screening of individuals for such cancers.

By way of further example, systems and methods according to the present teachings can be employed for the detection of head-and-neck cancer. Head and neck cancer generally refers to the presence of malignant tumors in the head and neck region, particularly in the upper aerodigestive tract and salivary glands. It has been reported that head and neck cancers account for about 3% of all cancers in the U.S. Over 70% of head and neck cancer cases are caused by high-risk Human Papilloma Virus (hr-HPV) infections, including, but not limited to, HPV-16, HPV-18, and HPV-11. Although many HPV infections clear up most of the time without leading to cancer, if an HPV infection persists in the body for an extended period and the HPV genome becomes integrated into the genome of the affected tissue, it may lead to cancer. Integration of hr-HPVs into the genome results in changes in genetics, proteomics, epigenetics, metabolomics, and the microbiota of the affected cells and tissues, thereby initiating and progressing tumorigenesis. Therefore, detecting the presence of hr-HPV genes or their transcripts, along with one or more orthogonal/phenotypic disease biomarkers that are altered in response to HPV infection, can enable more comprehensive and accurate detection, screening, and diagnosis. For example, integration of hr-HPVs into the genome of affected tissue results in the elevation of p16 protein levels. Detection of p16 transcript (mRNA) or protein as phenotypic biomarkers in liquid biopsy (i.e., saliva or blood) before a visible tumor, indicates that tumorigenesis events are initiated, and the patient either has cancer or requires close monitoring for early detection of cancer. Definitive diagnosis can be done through imaging (e.g., MRI, PET scan). Healthcare providers may recommend more intensive screening for certain high-risk groups of individuals, such as those with a family history of head-and-neck cancer, those with possible HIV infections based on age group and other activities, and those who engage in heavy smoking and drinking alcoholic beverages. In 2023, 23 million Americans were reported to be at high risk of developing head-and-neck cancer.

By way of example, for the detection of the head-and-neck cancer, a saliva specimen derived from an individual can be analyzed, e.g., in a manner disclosed herein, for the detection of HPV in the specimen, e.g., via detection of one or more target genomic nucleotide sequences of the HPV, and/or via detection of mRNA associated with transcription of the HPV genome in a host cell. By way of example, in various embodiments, the detection of an mRNA associated with the HPV and an mRNA, or a miRNA biomarker associated with a cellular process dysregulated by HPV infection can be utilized for the identification of an individual who is at the risk of having head-and-neck cancer. Alternatively, or in addition, a combined detection of HPV and a protein biomarker, such as p-16 protein can indicate that an individual is at the risk of having head-and-neck cancer.

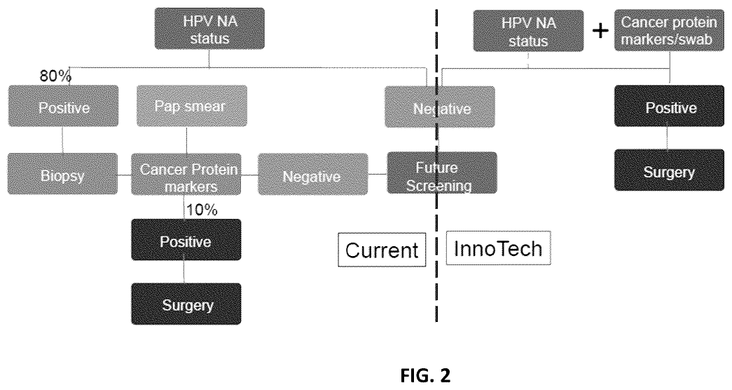

As noted above, in various embodiments, the present teachings can be employed for the detection of cervical cancer. By way of further illustration, FIG. 2 shows an example of a conventional workflow for the detection of cervical cancer and a workflow according to an embodiment of the present teachings. The conventional workflow depicted on the left side includes testing a patient's sample for the presence of the HPV and if the result in positive, performing a test for the detection of cancer protein biomarkers. If the cancer protein markers are detected, the presence of cancer is verified and treatment options are considered, otherwise, no action is taken, and future screening is recommended. Further, conventionally, a Pap smear test can be performed to extract cells from the surface of the cervix. The extracted cells can be examined for detection of cervical cancer or changes that may lead to cervical cancer. The cells can also be examined for the presence of cancer protein biomarkers. However, only in about 10% of cases, the biopsy performed after a positive HPV test, or the results obtained via a Pap smear test led to a positive indication of cancer. In other words, conventional approaches lead to significant unneeded tests with their concomitant cost and potential complications.

In contrast, in a workflow according to an embodiment of the present teachings depicted on the right side of FIG. 2, a single patient sample can be tested, e.g., on the same cartridge, for the presence of different classes of cancer biomarkers, e.g., genetic and protein components, where the negative result will indicate that no treatment protocol is needed and a positive result indicates the presence of cancer. In other words, in a single test, actionable results are provided.

In various embodiments, the detection of a target nucleotide sequence, e.g., a target genomic sequence and/or an mRNA or miRNA, is achieved via amplification of the target sequence followed by the detection of amplicons of the target nucleic acid sequence generated via such amplification. By way of example, in various embodiments, a Loop Medicated Isothermal Amplification (LAMP) is performed followed by a detection mechanism in which a CRISPR (Cas12a) system that can be selectively activated via the target nucleic acid sequence is employed to activate the fluorophore of a fluorophore-quencher probe (via incision of a link by the activated CRISPR enzyme, between the fluorophore and quencher) for exquisite selectivity.

Table 1 below lists examples of various types of biomarkers that have been reported for head-and-neck cancer.

TABLE 1
Examples of various types of biomarkers reported in head and neck cancer, cervical cancer

| | | Protein | | | Microbiomes |
|---|---|---|---|---|---|
| Disease | Gene | mRNA | miRNA | Metabolomics | Biomarker |
| Head & | HPV16 | P16 | miR-1268a | Phosphatidylserine | P melaninogenica |
| Neck | HPV18 | EGFR | miR-4505 | synthase 1 and | S mitis |
| Cancer | HPV35 | Ki-67 | | Lysophophatidylcholin | C gingivalis |
| | | | | Acytltransferase 1 | |
| Cervical | HPV16 | P16 | miR-34a | GSK3b | Non-lactobacilli |
| Cancer | HPV18 | Ki-67 | miR-9 | GAD1 | L-actobacillisiners |
| | HPV35 | Her2 | miR-21 | | |
| | | | miR-145 | | |
| | | | miR-375 | | |

As noted above, in various embodiments, an amplification technique, such as loop mediated amplification (LAMP) may be utilized, e.g., in a manner discussed above in connection with the previous embodiments, to amplify various nucleotide sequences, such as the genetic components of the virus, in a sample under test. Primers for loop mediated amplification (LAMP) of HPV 16, HPV 18 and HPV 31 have previously been reported (See, e.g., an article titled “Method for elucidation of LAMP products captured on lateral flow strips in a point of care test for HPV 16,” By Landaverde et al. published in Analytical and Bioanalytical Chemistry, volume 412, pages 6199-6209 (2020), which is herein incorporated by reference in its entirety; See, also, Detection assay for HPV16 and HPV18 by loop-mediated isothermal amplification with lateral flow dipstick tests,” by Kumvongpin et al, published in Molecular Medicine Reports, volume 15, Issue 5 (2017), which is also herein incorporated by reference in its entirety).

By way of example, Table 2 below presents primers published in “Assessing the performance of a loop mediated isothermal amplification (LAMP) assay for the detection and subtyping of high-risk subtypes of Human Papilloma Virus (HPV) for Oropharyngeal Squamous Cell Carcinoma (OPSCC) without DNA purification,” by Rohtensky et al., BMC Cancer volume 18, Article number: 166 (2018).

TABLE 2

| | SEQ | |
|---|---|---|
| | ID NO: | |
| HPV16 | | |
| F3 | 1 | TCGGTTGTGCGTACAAAG |
| B3 | 2 | AGCCTCTACATAAAACCATCC |
| FIP | 3 | TGGGGCACACAATTCCTAGT*CACACACGTAGACATT |
| | | CGT |
| BIP | 4 | TCAGAAACCATAATCTACCATGGC*ATTACATCCCGT |
| | | ACCCTCTT |
| LF | 5 | CCCATTAACAGGTCTTCCAAAGT |
| LB | 6 | CCTGCAGGTACCAATGGGG |
| HPV18 | | |
| F3 | 7 | AACGACGATTCCACAACA |
| B3 | 8 | CAACCGGAATTTCATTTTGG |
| FIP | 9 | GTCTTTCCTGTCGTGCTCGGT*AGCTGGGCACTATAG |
| | | AGG |
| BIP | 10 | CGACGCAGAGAAACACAAGT*CTCTAAATGCAATACA |
| | | ATGTCTTG |
| LF | 11 | CAGCACGAATGGCACTGG |
| LB | 12 | TATTAAGTATGCATGGACCTAAGGC |
| HPV31 | | |
| F3 | 13 | AGAAGAAAAACAAAGACATTTGG |
| B3 | 14 | CTCCTCATCTGAGCTGTC |
| FIP | 15 | GTCTTCTCCAACATGCTATGCA*GAAACGATTCCACA |
| | | ACATAGG |
| BIP | 16 | GAGAAACACCTACGTTGCAAGA*GGGTAATTGCTCAT |
| | | AACAGTG |
| LF | 17 | ACGTCCTGTCCACCTTCCT |
| LB | 18 | TGTGTTAGATTTGCAACCTGAGGCA |
| HPV35 | | |
| F3 | 19 | TGCATGGAGAAATAACTACATTG |
| B3 | 20 | CGCCTCACATTTACAACAG |
| FIP | 21 | TGTCACACAATTGCTCATAACAGTA*CAAGACTATGT |
| | | TTTAGATTTGGAAC |
| BIP | 22 | GCTCAGAGGAGGAGGAAGATAC*GACGTTACAATATT |
| | | ATAATTGGAGG |
| LF | 23 | TATTGACGGTCCAGCTGGACAA |
| LB | 24 | GGACAAGCAAAACCAGACA |

*Denotes a TTTT linker

Other nucleic acid amplification strategies such as Recombinase Polymerase Amplification (RPA) combined with Exonuclease (Exo) assay (herein referred to as RPA-Exo assay) can also be employed for rapid and highly specific amplified detection of target nucleic acid sequences. By way of an example, RPA-Exo is described in an article; titled “Rapid detection of SARS-COV-2 by low volume real-time single tube reverse transcription recombinase polymerase amplification using an exo probe with an internally linked quencher (exo-IQ) by Ole Berhmann, Iris Bachmann, Martin Spiegen, Marina Schramm, Ahmed Abd El Wahed, Grehrad Dobler, Gregory Dame, and Frank T Hufert, which is herein incorporated by reference in its entirety.

Briefly, RPA can be employed as an isothermal amplification alternative to the conventional polymerase chain reaction (PCR). Unlike PCR, which requires the use of a thermal cycler, RPA functions optimally with the temperature range of 37-42° C. Consequently, it can be executed using simpler and more cost-effective equipment. The RPA process relies on three key enzymes, a recombinase, a single-stranded DNA-binding protein (SSB), and a strand-displacing polymerase. Recombinases have the capability to pair oligonucleotide primers with homologous sequences in double-stranded DNA. SSBs bind to displaced DNA strands, thereby preventing the displacement of primers. Subsequently, the strand-displacing polymerase initiates DNA synthesis at the site where the primer has bound to the target DNA. Utilizing forward and reverse primers, an exponential DNA amplification is initiated if the target sequence is present. At the optimal temperature (37-42° C.), the reaction proceeds swiftly, resulting in the specific amplification of DNA from a few target copies to detectable levels, typically within about 10 minutes. This allows the rapid detection of DNA, RNA, as well as short length aptamer DNA. Incorporating exonuclease III facilitates the utilization of an exo probe for real-time, fluorescence detection akin to real-time PCR. Fluorescent signals generated by RPA-Exo can be detected using optical devices equipped with appropriate fluorescence filters. Additionally, lateral flow strip detection can be employed if both endonuclease IV and an nfo probe are utilized. With the addition of a reverse transcriptase that operates that 37-42° C., RNA can be reverse transcribed, and the resultant cDNA can be amplified, all in a single step. RPA reactions can be multiplexed via introduction of additional primer/probe pairs, enabling the detection of multiple analytes or an internal control within the same reaction mixture.

In various embodiments, the detection of protein biomarkers can be achieved using a variety of techniques known in the art. By way of example, an antibody that is tagged with a fluorescent probe and exhibits specific binding to a target protein can be utilized for the detection of the target protein.

Further, a novel assay for the detection of a target protein is herein described (which is herein referred to as Immune-RPA or as its abbreviated form iRPA), which can be employed in various embodiments. However, the use of such a method for the detection of a target protein is not restricted to the applications disclosed herein. Rather, it has general applicability for the detection of a target protein in a sample, e.g., a biological sample.

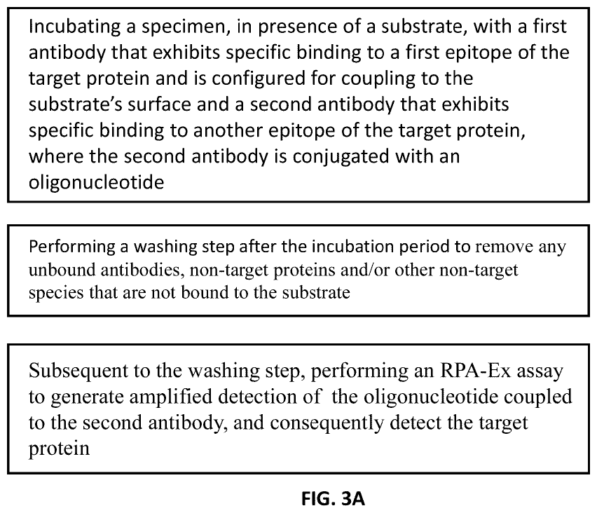

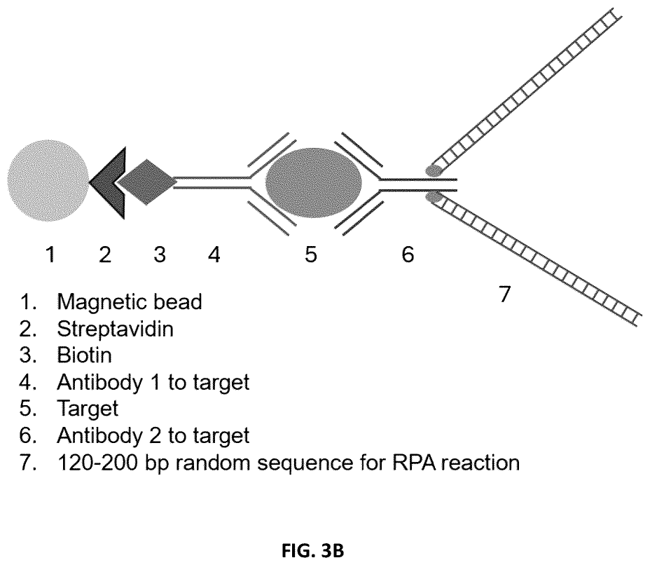

With reference to the flow chart of FIG. 3A and the diagram of FIG. 3B, such an assay for the detection of a target protein includes incubating, in presence of a substrate, a specimen with a first antibody that exhibits specific binding to a first epitope of the target protein and is configured, e.g., via biotinylating (See, e.g., biotinylated-antibody 1), to the substrate, e.g., a substrate labeled with Streptavidin (See, e.g., magnetic bead 1 functionalized with Streptavidin 2), and a second antibody (See, e.g., antibody 2) that exhibits specific binding to another epitope of the target protein, where the second antibody is conjugated with an oligonucleotide (See, e.g., conjugation of antibody 2 to a random RPA sequence of 120-200 nucleotide base pairs). In addition, in various embodiments, PBS-Tween with added salmon-sperm DNA is added to the reaction mixture.

The reaction is generally carried out at room temperature for a time period, e.g., in a range of about 30-40 minutes. When the target protein is present in the specimen, the incubation of the specimen as discussed above can result in binding of the target protein to the first and the second antibodies, with the first antibody being coupled to the substrate (e.g., via binding of the biotin to the streptavidin). Subsequent to the incubation step, a washing step can be performed to remove any unbound antibodies non-target proteins, and/or other species that are not bound to the substrate.

By way of example, and with particular reference to FIG. 3B, when the capture substrate constitutes magnetic beads, the reaction products are subjected to a magnet field to capture the complex of the magnetic beads and associated antibodies, conjugated oligonucleotide sequences and the target protein. Any unbound components are washed with PBS-Tween.

Following the washing step, RPA-Exo reaction components are added to amplify the oligonucleotide sequence and obtain a fluorescent signal for identification of the presence of the target protein.

In various embodiments, the above method immune-RPA (iRPA) significantly increases the signal for small amounts of protein present in the specimen.

By way of example, the above iRPA protein detection assay can be used for detection of proteins at femtomole and attomole levels. By way of example, a target-selective antibody is labelled with biotin to be bound to a Streptavidin treated tube or plate or labelled with a Streptavidin Dynabeads. Another matched antibody for the same target that has an epitope other than the first antibody is conjugated with a random oligonucleotide of 150-200 nucleic acids that is designed to be used in an RPA-Exo reaction of RPA primers and labelled Exo probes. In one embodiment an antibody for p16 is labelled with Streptavidin Dynabeads. Another matched p16 antibody is labelled with random RPA oligonucleotide. The two antibodies and p16 protein are mixed for 30-45 minutes. Each antibody has a separate epitope for binding to p16. Following incubation, a magnetic field is introduced and a wash step is performed to remove any unbound antibodies and other unbound components. A reaction mixture for RPA-Exo is introduced and following 10 minutes the reaction is read using a fluorescent reader.

In various embodiments, a system for point-of-care detection of cervical cancer is provided, which includes a sensor, e.g., an electrochemical or optical sensor, that is configured for the point-of-care detection of at least one of high-risk HPV 16, HPV 18, and HPV31 nucleic acids as well as the detection of at least one cervical cancer protein biomarker, such as P16 tumor suppressor protein also known as p16INK4a/CDKN2A (herein referred to as P16) and/or Ki-67. In some embodiments, the system may be configured to detect, in addition to at least one of the high-risk nucleic acids and at least one of the P16 and Ki-67 proteins, at least one of the methylated DNA biomarkers, ASCL1 or LHX8. The concurrent detection of two or more of the genetic, the protein and methylated DNA markers associated with cervical cancer can inform a clinician whether an individual with positive HPV is progressing to cervical cancer.

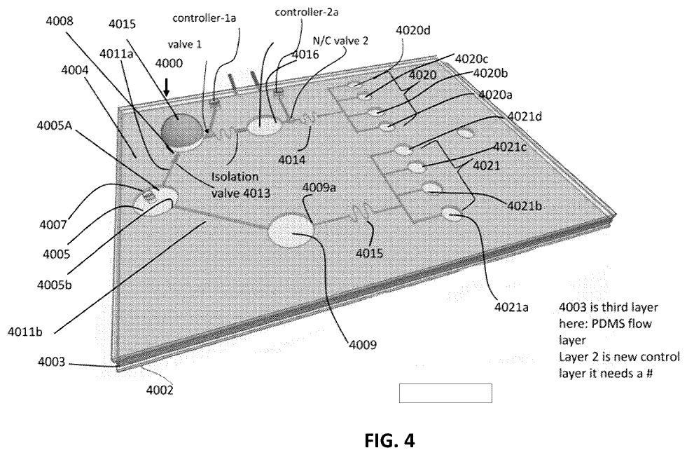

In various embodiments, a cartridge can be utilized for the multiplexed detection of orthogonal classes of biomarkers in a specimen. By way of example, cartridges disclosed in U.S. Pat. No. 11,541,393 (herein “the '393 patent”) titled “Systems, apparatus, and method for detecting pathogens,” which is assigned to the assignee of the present application and which is herein incorporated by reference in its entirety, can be configured based on the present teachings for the multiplexed detection of multiple orthogonal classes of biomarkers. For example, the cartridge depicted in FIG. 19A of this patent, which is reproduced herein as FIG. 4, can be configured based on the present teachings for the detection of multiple orthogonal classes of biomarkers.

By way of example, the cartridge depicted in FIG. 4 can be configured to include one bank of detecting elements that is configured to detect at least one nucleotide sequence associated with the detection of the HPV virus and another bank of the detecting elements (herein also referred to as sensors) that is configured to detect phenotypic, regulatory, metabolomic and/or microbiome-derived biomarkers that can be dysregulated due to HPV infection. For example, the detecting elements can be electrochemical sensors in which the working electrode has been functionalized by agents, such as, oligonucleotides, antibodies, aptamers, SOMAmers, raptomers, megastars or combinations thereof. By way of example, in some such embodiments, the electrochemical sensors for detection of the genetic components of the HPV virus can be functionalized with an oligonucleotide that exhibit specific binding to the target genetic components of the virus and the electrochemical sensors for the detection of the protein biomarkers can be functionalized with antibodies that exhibit specific binding to those proteins. In some embodiments, one bank of detectors can include a plurality of detectors each of which is configured for the detection of a different genetic biomarker associated with the HPV virus and the other bank of detectors can include a plurality of sensors each of which is configured for the detection of a different protein biomarker associated with cervical cancer. In this manner, in such embodiments, a single cartridge can provide not only multiplexed detection of genetic and protein biomarkers, but also provide in each category (i.e., detection of genetic or protein markers) the ability to detect multiple different members of that category, thereby enhancing sensitivity and specificity of the device.

More specifically, the disposable cartridge 4000 includes three layers 4002 (bottom layer), 4003 (middle layer), and 4004 (top layer) that are assembled to collectively form a cartridge frame. In this embodiment, the top and bottom layers are formed of optically transparent materials, e.g., glass or an optically clear polymeric material. In some embodiments in which the top layer is formed of glass, the inner surface of the glass may be coated with a polymeric coating (e.g., a coating of PDMS) at least in areas of the inner glass surface that may come into contact with portions of a sample under analysis. In some such embodiments, the entire inner coating of a top glass layer may be coated with a layer of a suitable polymeric coating.

In this embodiment, the middle layer includes a sample-receiving well 4005 that can receive, via an inlet port 4007, a sample for analysis. The cartridge 4000 can be configured for analysis of a variety of different biological specimens. In general, the cartridge 4000 can be configured for diagnostic analysis of liquid biopsy samples. Some examples of samples that can be analyzed using the cartridge 4000 include, without limitation, blood and saliva.

The cartridge 4000 further includes two wells (reservoirs) 4008/4009 in one of which a buffer for processing (preparing) a portion of the received specimen for detection of one or more target nucleotide sequences, when present in the received sample, is stored (herein referred to for brevity as the “genetic buffer)) and in the other a buffer for processing (preparing) another portion of the received specimen for detection of one or more target proteins is stored (herein referred to for brevity as the “protein buffer”).

For case of description, it is assumed that in this embodiment, the well 4008 stores the genetic buffer and the well 4009 stores the protein buffer. A fluid channel 4011a fluidly connects the sample-receiving well 4005 to the buffer well 4008 and another fluidic channel 4011 b fluidly connects the sample-receiving well to the buffer well 4009. An isolation valve 4013 inhibits the backflow.

The specimen portions received in the reservoirs 4008/4009 come into contact with the genetic and protein buffers, respectively. The buffer reservoir 4008 can be in the form of a blister pouch 4015 in which the genetic buffer is stored. The blister pouch 4015 includes a flexible membrane 4015a forming an enclosure within which the buffer is stored. A separation membrane 4015b is disposed within the blister pouch over an internal puncture arm 4015c. The blister pouch can be activated to cause the liquid stored within the pouch to be released by pressing on the flexible membrane 4015a such that the increase in the liquid pressure within the blister's enclosure can cause the crushing of the internal puncture arm, via pressure exerted thereon by the separation membrane 4015 b, thereby releasing the liquid from the pouch.

In this embodiment, a pneumatically-controlled valve 1 coupled to the blister pouch 4015, and controlled via a controller 1a, facilitates the transfer of the liquid released from the blister pouch 4015 to a downstream amplification reservoir (well) 4016 for amplifying nucleotide sequences (e.g., DNA/RNA sequences) in the liquid released from the blister, when that nucleotide sequence is present in a specimen collected from a subject. As noted above, the amplification of the nucleotide sequences can be achieved using a variety of different amplification modalities. For example, in some embodiments, isothermal amplification methods can be used while in others amplification/detection methods, such as RPA-Ex, can be employed.

By way of example, in this embodiment, the amplification well 4016 can contain one or more reagents (such as primers) suitable for performing isothermal amplification of a target nucleotide sequence, when that pathogen is present in the sample. By way of example, a genetic buffer contained in the buffer reservoir can include, among other reagents, a reagent for lysing a viral particle so as to release one or more RNA/DNA segments. Such RNA/DNA segments can then undergo isothermal amplification in the amplification well. A heating/cooling element, such as Peltier device and its associated thermistor, can be incorporated in the cartridge for maintaining the temperature of the genetic buffer at a desired value. After amplification of the nucleotide sequences is completed, a pneumatically-controlled valve 2 can be activated via its associated controller 2a to transfer the amplified sample, via passage through a mixing element 4014 that is implemented as a serpentine channel, to a bank of sensors 4020, each of which is configured to detect a different nucleotide sequence. The passage of the sample through the mixing element 4014 further facilitates the preparation of the sample for detection of the target nucleotide sequences, if any, therein. For case of description, the liquid exiting the mixing element is herein referred to as a processed sample.

More specifically, in this embodiment, the bank of detecting elements 4020 includes four sensors 4020a, 4020b, 4020c, and 4020d, each of which is configured to detect a different target nucleic acid sequence (e.g., RNA and/or DNA strands). By way of example, in some implementations, each of the detecting elements 4020 can be implemented as an electrochemical sensor functionalized for detection of a target nucleotide sequence.

With continued reference to FIG. 4, the protein buffer reservoir 4009 is fluidly connected to the sample-receiving reservoir 4005 to receive a portion of a sample collected from an individual via a fluidic channel 4011 b. The interaction of the received sample portion with the protein buffer can generate a processed sample suitable for introduction into a bank of detectors 4021 for detection a plurality of proteins associated with the target pathogen, when present in the sample collected from the subject. More specifically, in this embodiment, the bank of detecting elements 4021 includes four sensors 4021 a, 4021 b, 4021 c, and 4021 d, each of which is configured to detect a different target protein. In some implementation, there may only one sensor for detecting a single target protein or more sensors for detecting additional target proteins. By way of example, in this embodiment, each of the detecting elements can be implemented as an electrochemical sensor functionalized for detection of a different target protein, e.g., via functionalizing the working electrode of each electrochemical sensors with a different antibody that exhibits specific binding to a different target protein.

Although in this cartridge, no amplification of a signal associated with a target protein is implemented, in other cartridges, various techniques such as the iRPA technique discussed herein can be utilized for amplifying the signal associated with the detection of a target protein.

In other embodiments, in addition to or instead of electrochemical detection, one or more of the detecting elements may be configured for optical detection of a target protein and/or a target nucleotide sequence. By way of illustration, for example, fluorophore-quencher probes can be employed for detection of target nucleotide sequences using, e.g., RPA-Exo assay.

A reader unit (herein also referred to as an analyzer), such as those discussed in the '393 Patent and modified as informed by the present teachings, can receive detection signals from the cartridge and process those signals to detect various biomarkers of interest.

In some embodiments, the electrochemical detection of a nucleic acid sequences (e.g., associated with viral particles, such as HPV) can be performed using the techniques described in “Electrochemical strategy for low-cost viral detection,” by Zamani et al., ASC Cent. Sci. 2021, 7, 963-972, which is herein incorporated by reference in its entirety. Briefly, in such an approach, DNA-modified gold leaf electrodes are used to monito HPV DNA-activated Cas12a endonuclease activity. The gold lead electrodes were functionalized with methylene blue-tagged oligonucleotides, Cas12a was used to detect a plasmid containing the HPV genetic sequence. The guide RNA (gRNA) of the Cas12a enzyme was engineered to recognize the p24 locus of the HPV gag gene. The activated enzyme causes the cleavage of the oligonucleotide attached to the gold surface, which releases methylene blue, a well-established redox mediator, causing a change in an electrochemical signal obtained via cyclic voltammetry. This strategy does not allow multiplexing within the same well, but spatial multiplexing can be done.

Further, in some embodiments, one or more sensors of a cartridge according to various embodiments can be functionalized with an agent that exhibits specific binding to a protein biomarker for the detection of that protein in the sample. By way of example, antibodies that exhibit specific binding to the p16 protein are commercially available and can be utilized to functionalize a working electrode of an electrochemical sensor according to various embodiments. For example, Abcam Human CDKN2A/p16INK4a (ab227903) antibody can be utilized for this purpose.

Further, in some embodiments, one or more sensors of a cartridge according to the present teachings can be configured for detection of one or more epigenetic changes associated with a disease in conjunction with point mutations and/or protein expression. By way of example, such an epigenetic change can correspond to methylation of a gene, such as RUNX3, SFRP1, WIF1, PCDH10, DKK2, DKK3, TMEFF2, OPCML, and SFRP2 that have higher methylation levels in tumor compared to normal tissue. In addition, K-Ras mutations are found in more than 40% of colon cancers. https://doi.org/10.3389/fonc.2021.697409.

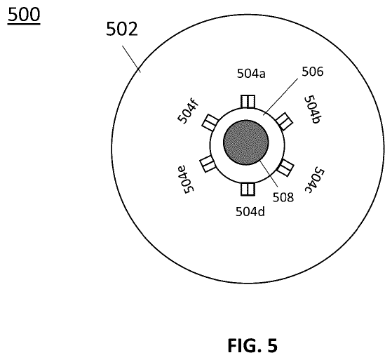

By way of further examples suitable devices for performing methods according to various embodiments, FIG. 5 shows a top schematic view of a system 500 that includes a housing 502 in which six emitter/detector pairs 504a, 504b, 504c, 504d, 504e, and 504f are disposed, which are distributed about an opening 506 in which a vial 508 can be inserted such that a specimen disposed in the vial can be exposed to the radiation emitted by the emitters of the emitter/detector pairs. The detector of each emitter/detector pair can detect fluorescent radiation emitted from the specimen in the vial, as discussed below. By way of example, in this embodiment, the emitter/detector pairs allow multiplexed detection of six different target nucleotide sequences using six different fluorophore/quencher probe pairs. In various embodiments, optical filters placed in front of the detectors of the emitter/detector pairs can be utilized to ensure that each detector would detect fluorescent radiation at a different wavelength.

More specifically, in this embodiment, each of the emitter/detector pairs can be employed for exciting and detecting fluorescent radiation from a different nucleotide sequence, though the system can also be employed for detection of different proteins (e.g., using fluorescent-tagged antibodies) or a combination of one or more proteins and one or more nucleotide sequences. In some embodiments, one or more of the emitter/detector pairs can be used for the detection of one or more target proteins and one or more of the other emitter/detector pairs can be used for the detection of one or more target genome sequences, one or more mRNAs or miRNAs, etc.

The multiplexing of the detection of six target biomarkers (e.g., proteins) can be achieved using each of the following fluorophores for the detection each target protein: FAM (excitation: 430 nm; detection: 480 nm), HEX/VIC (excitation: 470 nm; detection: 515 nm), ROX (excitation: 545 nm; detection: 565 nm), Cy5 (excitation: 575 nm, detection: 610 nm), Cy5.5 (excitation: 628 nm, detection: 670 nm), Ato425 (excitation: 682 nm, detection: 725 nm).

The fluorescent signals generated by the detectors of the emitter/detector pairs can be processed, e.g., using an on-board process to identify whether target nucleic acid sequences are present in the sample.

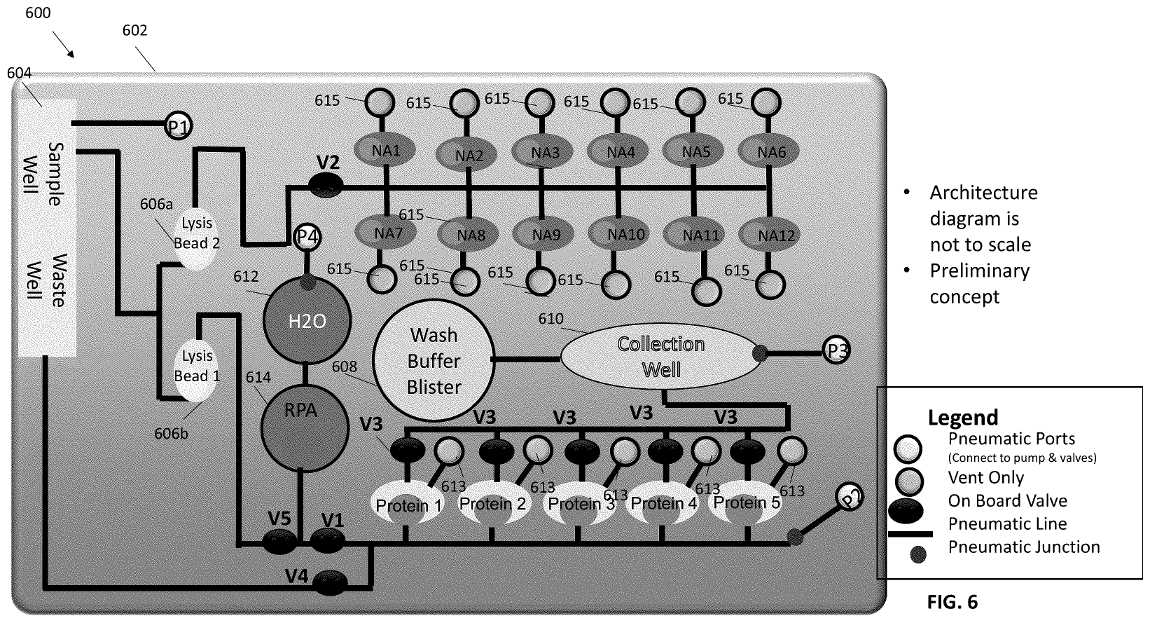

By way of further illustration, FIG. 6 is a schematic view of a cartridge 600 according to some embodiments, which includes a frame 602 having a sample well, which can receive a specimen. A pneumatic port P1 can be activated (e.g., under the control of a controller, not shown in this figure) to apply a positive pressure to the sample within the sample well to distribute the sample between a lysis chamber 606a (herein also referred to as the nucleic acid lysis chamber) that contains lyophilized reagents for processing the specimen for nucleic acid detection and another lysis chamber 606b (herein also referred to as the protein lysis chamber) for processing the specimen for detection of one or more target proteins.

The cartridge 600 further includes a plurality of protein detection wells Protein 1, Protein 2, Protein 3, Protein 4, and Protein 5. Two valves V1 and V5 regulate the flow of the sample from the protein lysis chamber 606b to the plurality of protein detection wells. The cartridge further includes a plurality of nucleic acid (NA) detection wells NA1, NA2, NA3, NA4, NA5, NA6, NA7, NA8, NA9, NA10, NA11 and NA12. The flow of the sample between the nucleic acid lysis chamber is regulated via a valve V2.

In one mode of operation, the valve V1, V5, V2 and V4 are closed as the sample is transferred, under the influence of a positive pressure provided by a pneumatic port P1, from the sample well 604 to the nucleic acid and protein lysis chambers 604a and 604b. Subsequently, the valves V5 and V1 are opened while valves V2, V4 and V6 remain closed to transfer the sample portion in the protein lysis chamber to protein detection wells Protein 1-Protein 5. In this embodiment, the protein detection wells contain a plurality of magnetic beads that are functionalized with Streptavidin and two antibodies each exhibiting specific binding to one epitope of a target protein, where one of the antibodies is biotinylated for binding to the Streptavidin of the magnetic beads and the other antibody is conjugated with one or more 120-200 bp random nucleotide sequences.

After a predefined incubation period, a wash buffer blister pack 608 provided in the cartridge frame can be punctured (via a controller unit into which the cartridge is introduce following the introduction of the sample into the cartridge) so as to cause a wash buffer contained therein to flow to a wash buffer collection well 610. A plurality of valves V3 positioned between each of the protein detection wells and the wash buffer collection well 610 can be opened and a pneumatic port P5 can be activated to pressurize the wash buffer within the collection well 610 to cause its flow to the protein detection wells. Subsequently valves V1 is closed and valve V4 is opened to transfer the wash buffer, under the influence of a positive pressure provided via the pneumatic port P4, from the protein detection wells to the emptied sample reservoir, which acts as a waste well at this stage of operation. During the wash step, a magnetic field is applied (via a controller, not shown in this figure) to the protein detection wells to force the magnetic beads toward the bottom of the wells, thereby inhibiting them from exiting the protein detection wells.

A plurality of hydrophobic vents 613 prevent the interference of bubbles that may be formed with ingress and egress of liquid into and from the protein detection wells.

Subsequent to the completion of the wash step, valve V4 is closed and a blister pack 612 containing water is punctured and a pneumatic port P6 is activated to apply positive pressure to the water released from the blister pack to transfer the water to a well 614 in which lyophilized reagents for performing an RPA-Exo assay is stored, where the water reconstitutes the lyophilized reagents. This is followed by opening valves V6 and V1 to transfer the RPA-Exo reagents to the protein detection wells. Each protein detection well can be configured, via the selection of the antibodies, for the detection of a different target protein. The fluorescent emitted by the fluorophore of the RPA probe can be detected via detectors provided in a reader unit and the signals can be analyzed reader via firmware provided on the reader unit or remotely to identify the presence of one or more target proteins within the sample under analysis. By way of example, the fluorescent radiation emitted from each protein detection well can be detected periodically, e.g., every one minute, to generate a plurality of fluorescent detection signals that can be analyzed for the detection of the target proteins.

In some embodiments, at least one of the protein detection wells can be configured for multiplexed detection of two or more proteins, e.g., via storing fluorophore-tagged antibodies that exhibit specific binding to two or more target proteins and having fluorophore probes that emit light at different wavelengths when excited.

Subsequently, during the process of fluorescence detection from the protein detection wells, valve V2 is opened, while valve V5 remains closed to transfer the sample portion in the lysis chamber 606a to the plurality of nucleic acid detection wells NA1 to NA12. NA1 through NA12 wells can contain lyophilized reagents for amplification of different target nucleic acid sequences, e.g., via isothermal amplification, as well as lyophilized reagents, such as CAS12a system and fluorophore-quencher probes. In various embodiments, each of the nucleic acid wells can be utilized for detection of up to 6 nucleic sequence targets using probes with six different fluorophore and quencher pairs (e.g., using the following fluorophores: FAM, HEX/VIC, ROX, Cy5, Cy5.5, and Ato425). The fluorescent radiation generated in each of the nucleic acid wells can be detected via a plurality of detectors provided in a reader device that is configured to receive the cartridge and can be analyzed to identify target nucleic acid sequences of interest.

Similar to the protein detection wells, each of the nucleic acid detection wells is coupled to one of a plurality of hydrophobic vents 615.

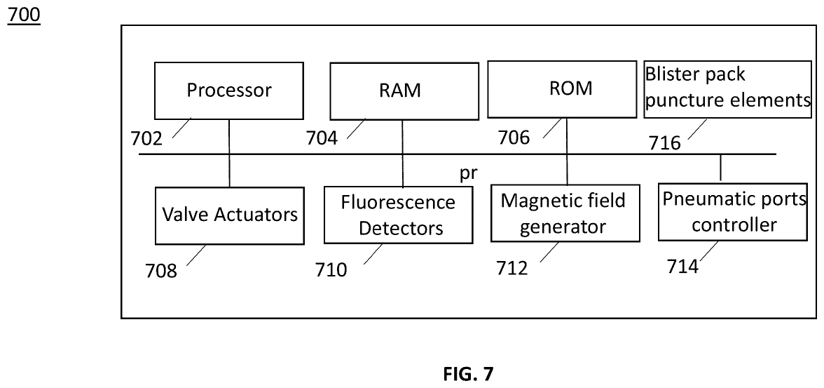

FIG. 7 a schematic diagram depicting various components of a controller/fluorescent 700 reader that can be employed to control cartridge 600 and detect the fluorescent radiation generated in various wells of the cartridge. The controller 700 includes a digital data processor 702, a random-access memory module (RAM) 704 and a read only memory (ROM) module 706, which operate under the control of the processor 702. The controller 700 further includes a plurality valve actuator 708, a plurality of fluorescence detectors 710, a magnetic field generator 712 and a pneumatic ports controller 714, a plurality of blister pack puncture elements 716, which also operate under the control of the processor 702.

A variety of available valve actuators, fluorescent detectors, magnetic field generators, and pneumatic ports controller can be used in various implementations of the controller. By way of example, and without limitation, the valve actuators can be in the form of spring-loaded Pogo pins that can be actuated to block a fluid channel to prevent passage of liquid through the channel. Again, by way of example, the magnetic field generator can be an electromagnet operating under the control of the controller that can be actuated to ensure that the magnetic beads within the protein detection wells are retained within the wells.

A set of instructions for operating the cartridge in accordance with various embodiments can be stored on the ROM module and can be transferred to the RAM module during execution to be executed via the processor. By way of example, the set of instructions can include the timings for opening and closing of various valves, activating various pneumatic ports, puncturing various blister packs, applying a magnetic field to the protein detection wells and detecting radiation via the fluorescent detectors. Further, in some embodiments, the ROM module can also include instructions for analyzing the fluorescent signals generated by the fluorescent detectors for identification of pathogens and/or biomarkers of interest.

With the ability to provide multiplexing in each chamber and spatial multiplexing, the cartridge will allow high performance multiplexing of multiple of not just the same class of biomarkers but of multiple different classes of biomarkers within the same all enclosed and easy to use cartridge with the user just placing the specimen on the cartridge.

As discussed above, the all-enclosed cartridge can have spatially separated chambers allowing multiplexing of multiple of genetic markers and multiplexing of multiple of protein, genomic, metabolomics or other markers. As an example, each of HPV 16, HPV 18, HPV 31, HPV 35 can be detected in a separate nucleic acid well, where each chamber contains lyophilized reagents for amplification of the specific target. Alternatively, the same nucleic acid well can be utilized for multiplexed detection of the above HPV strains with lyophilized reagents for each of them and probes with different fluorophore and quencher that are unique to each target. As an example, FAM, HEX, CY5, ROX and quenchers specific for each can be used for multiplexing.

Multiplexing of 120 biomarkers in a chip with 20 reaction chambers can easily be achieved. For such multiplexing, Recombinase Polymerase Amplification combined with Exonuclease assay at temperatures of 37-40 are performed. Exonuclease probes with fluorophore and quenchers for up to 6 different wavelengths can be incorporated such as FAM, HEX/VIC, ROX, CY5, CY5.5, Ato 425 with excitations at 430 nm, 470 nm, 535 nm, 575 nm, 628 nm, 682 nm, and detections of 480 nm, 515 nm, 565 nm, 610 nm, 67 nm, and 725 nm using LED light and photodiode detectors.

In some embodiment the innovative Immune-RPA (IRPA) is used in the cartridge for amplified detection of target proteins. For example, a p16-positive specimen can be introduced into the cartridge and pushed to the protein lysis chamber. After 1-5 minutes of incubation and mixing within the cartridge, the lysate is pushed to the i-RPA chamber where the Streptavidin-Dyna bead labeled, and the oligo labeled antibodies to p16 are present. A within cartridge mixing step such as push-pull will be carried out and after 30-40 minutes of reaction, a magnetic field will be activated. A blister pack within the cartridge will be punctured and will transfer wash buffer through the protein reaction chambers. The used wash buffer will be pushed back to the specimen chamber that is now empty and can serve as a waste chamber to save space. Another chamber with reaction reagents for the RPA-Exo assay will be initiated to push the reagents to the protein chamber. After 10 minutes, the presence or absence of target protein will be measured based on the fluorophore and quencher that was used for that specific target and incorporated in the Exo-probe.

The present teachings can provide a platform for detection of orthogonal classes of biomarkers not only for diagnosis of cancer, but also for diagnosis of other diseases associated with multiple classes of biomarkers, such as neurodegenerative, and infectious diseases.

By way of example, a novel differentially methylated RHBDF2 gene has been found in ALS patients compared to controls from cell-free DNA (cfDNA) isolated from Whole blood samples (See, e.g., Liquid biopsy: a new source of candidate biomarkers in amyotrophic lateral sclerosis,” by Mendioroz et al., published in Ann Clin Transl Neurol., 2018 Apr. 16: 5 (6): 763-768, which is herein incorporated in its entirety by reference)

Additionally, serum NfL is a clinically validated prognostic biomarker for ALS. (See, e.g., “Validation of serum neurofilaments as prognostic and potential pharmacodynamic biomarkers for ALS,” by Benatar et al., published in Neurology, 2020 Jul. 7: 95 (1): e59-e69, which is herein incorporated by reference in its entirety). Mutations in ALS predisposition genes such as A4V and SOD1 or a hexanucleotide repeat expansion in the C9ORF72 gene are also genotypic biomarkers of ALS. (See, “ALS biomarkers for therapy development: State of the field and future directions,” by Benatar et al., published in Muscle & Nerve, vol. 53, issue 2, pp. 169-182, which is herein incorporated by reference). A number of miRNAs and muscle miRNAs have been identified that their levels are either increased or decreased in ALS and they are being evaluated as diagnostic biomarkers of ALS. By way of example, miR-1825 and miR-1234-3p were consistently downregulated in the serum of sporadic ALS patients. Table 2 shows a list of diagnostic biomarkers in ALS. (Taken from “Serum microRNA in sporadic amyotrophic lateral sclerosis,” by Freischmidt et al., published in Neurobiol Aging, 2015 September: 36 (9): 2660. e15-20, which is herein incorporated by reference). Detection of two or more of these genotypic, phenotypic and epigenetic ALS biomarkers simultaneously can provide more timely and informed therapeutic decision making and improve or save lives of the patients.

TABLE 3
Potential circulating miRNA biomarkers in ALS.

| | | | Target/Signaling | |
|---|---|---|---|---|
| miRNA | Model | Change | Pathway | Functions |
| miR-206 | SOD1_{G93A} mice | ↑ | HDAC4 | miR-206 slowed progression of |
| | | | FGFBP1 | ALS by sensing MN damage and |
| | | | | promoting compensatory |
| | | | | regeneration of neuromuscular |
| | | | | synapses. |
| miR-155 | SOD1^{G93A} mice | ↑ | APOE pathway | Genetic ablation of miR-155 |
| | | | | reversed the proinflammatory |
| | | | | signature of both peripheral tissues. |
| DM_US209982831-1.119275.0032 | SOD1^{G93A} mice | ↑ | NF-κB pathway | miR-125b inhibition through A20 |
| miR-125b | | | A20 | protein protects MNs from death |
| | | | | induced by activating G93A. |
| miR-193b-3p | SOD1^{G93A} mice | ↑ | mTOR | Downregulation of miR-193b-3p is |
| | NSC-34 cell | | TSC1 | essential for cell survival by |
| | | | | targeting TSC1-mTOR signaling in |
| | | | | NSC-34 cells. |
| miR-375-3p | wobbler mouse | P20:↑ | p53 | After downregulating miR-375-3p |
| | | P40:↓ | NDRG2 | expression, inefficient inhibition of |
| | | | | p53 results in overexpression of |
| | | | | NDRG2, increasing ROS |
| | | | | generation and creating a vicious |
| | | | | cycle. |
| miR-18b-5p | fALS patient | | Hif1α | miR-18b-5p induced HIF1α, which |
| | SOD1^{G93A} mice | | Mef2c | increased the expression of Mef2c. |
| | NSC-34 cell | | miR-206 | Mef2c upregulated miR-206 as a |
| | | | Mctp1 | transcription factor. Inhibition of |
| | | | Rarb | mctp1 and RARB as miR-206 |
| | | | | targets induced intracellular |
| | | | | Ca²⁺ levels and reduced cell |
| | | | | differentiation, respectively. |
| miR-183-5p | SOD1^{G93A} mice | | PDCD4 | miR-183-5p regulated cell |
| | | | RIPK3 | apoptosis |
| | | | | by targeting PDCD4 and |
| | | | | necroptosis by RIPK3, respectively. |
| miR-124 | SOD1^{G93A} mice | | | Upregulation of miR-124 is related |
| [86] | NSC-34 cell | | | to the degeneration of mSOD1 |
| | | | | MNs, the deregulation of neuro- |
| | | | | immune crosstalk and the |
| | | | | imbalance of homeostasis. |
| miR-105 | sALS patient | ↓ | PRPH, | Downregulation of miR-9 and |
| miR-9 | | | INA | miR-105 in sALS might contribute |
| | | | | to the loss of intermediate filament |
| | | | | stoichiometry, ultimately leading to |
| | | | | intermediate filament aggregation |
| miR-126-5p | SOD1^{G93A} mice | ↓ | Sema3A | and eventually neuronal death. |
| levels | facilitated axon | | NMJ | Downregulation of miR-126-5p |
| | degeneration | | | Downregulation of miR-1825 |
| | and disruption. | | | caused translational upregulation of |
| miR-1825 | sALS patient | ↓ | CASP3 | TBCB, which might lead to |
| | | | | depolymerization and degradation |
| | | | | of TUBA4A. |

By way of another example, Alzheimer's disease (AD) is characterized by accumulation of the amyloid-β (Aβ) peptide precursor protein (APP). miR-346 has been found to regulate AB production. (See, e.g., “Novel upregulation of amyloid-β precursor protein (APP) by microRNA-346 via targeting of APP mRNA 5′-untranslated region: Implications in Alzheimer's disease,” by Long et al., published in Mol Psychiatry. 2019 March: 24 (3): 345-363, which is herein incorporated by reference in its entirety). Other studies show that a panel of 10 miRNAs (hsa-mir-107, hsa-mir-26b, hsa-mir-30c, hsa-mir-34a, hsa-mir-485, hsa-mir200c, hsa-mir-210, hsa-mir-146a, hsa-mir-34c, and hsa-mir-125b) are deregulated early in AD. (See, e.g., “Systemic review of miRNA as biomarkers in Alzheimer's disease,” by Swarbrick et al., published in Mol Neurobiol Feb. 8, 2019, which is herein incorporated by reference in its entirety)”

Several blood-based biomarkers have also been identified with altered gene expression in early stages of AD. These include blood RAB7A, NPC2, TGFB1, GAP43, ARSB, PER1, GUSB, MAPT, GSK3B, PTGS2, APOE, BACE1, PSEN1, and TREM2. (See, e.g., “Blood biomarkers for memory: toward early detection of risk for Alzheimer disease, pharmacogenomics, and repurposed drugs,” by Niculescu, published in Mol Psychiatry, 25 (8): 1651-1672, 2020) Blood metabolite signatures have also been identified as potential biomarkers for early detection of AD. Specifically, sphingolipids are thought to be biologically relevant biomarkers for the early detection of AD. Our approach of detecting two or more of the gene or protein expression, miRNAs or metabolites that are altered in AD can provide early and accurate detection of this devastating disease.

By way of another example, RT-PCR, Isothermal PCR and other nucleic acid (NA) amplification tests have been developed for the detection of SARS-COV-2. Antigen detection assays such as lateral flow assays (LFA) have also been developed. The lab based and some of the point of care tests (POCT) for detection of SARS-COV-2 provide high sensitivity and detect the pathogen at a low limit of detection. However, clinical and public health challenges have emerged as some patients continue to test positive for SARS-COV-2 weeks or months after infection. Thus, the NA tests alone cannot differentiate between infection and infectiousness. On the other hand, the protein assays alone, generally detecting the Nucleocapsid Protein, have a low sensitivity and high rate of false negatives. Therefore, simultaneous detection of both proteins and nucleic acids can provide early and accurate detection of SARS-CoV-2 and provide information about infection state and infectiousness of the patient. Additionally, it is important to understand the host immune response to pathogen. After infection with the SARS-COV-2 or a COVID-19 vaccine, host-response antibodies are generated in 2-3 weeks. There antibodies may remain in blood for many months.

Simultaneous and orthogonal detection of the antibody response to infection or vaccination, in conjunction with evaluation of the presence of infectious agent can provide a comprehensive timely and accurate results on the infectious and infectiousness status.4

Those having ordinary skill in the art will appreciate that various changes can be made to the above embodiments without departing from the scope of the invention.

1. A method for screening an individual for head and neck cancer, comprising:
acquiring a saliva sample from the individual,
analyzing the saliva sample to detect one or more of HPV genomes and at least one of a phenotypic biomarker, a regulatory biomarker, a microbiome-derived biomarker, and a metabolomic biomarker dysregulated due to HPV infection,
wherein detection of both of the HPV and at least one of said biomarkers indicates the individual is at risk of head and neck cancer.

2. The method of claim 1, wherein the phenotypic biomarker comprises any of a protein and an mRNA.

3. The method of claim 1, wherein the regulatory biomarker comprises a miRNA.

4. The method of claim 1, wherein the step of analyzing the salvia sample to detect the HPV comprises detecting at least one nucleotide target sequence associated with the HPV genome.

5. The method of claim 1, wherein the step of analyzing the saliva sample to detect the HPV comprises detecting at least an mRNA associated with transcription of the HPV genome in an infected cell.

6. The method of claim 1, wherein any of said at least one phenotypic biomarker is associated with a cellular molecular process affected by the HPV infection.

7. The method of claim 1, wherein said at least one protein biomarker comprises p-16 protein and EGF receptor.

8. The method of claim 1, further comprising triaging the individual for further confirmatory diagnosis such as MRI imaging if the individual is found to be at risk of head and neck cancer.

9. An assay for detecting a target protein in a sample, comprising:
incubating, in presence of a substrate, the sample with a first antibody exhibiting specific binding to a first epitope of a target protein, and a second antibody exhibiting specific binding to a second epitope of the target protein to form an incubating mixture of the sample and the antibodies, wherein the first antibody is further configured for coupling to the substrate and the second antibody is conjugated to a nucleotide sequence,
subsequently, performing a wash step of incubating mixture to remove components thereof that are not bound to the substrate,
using an RPA-Exo assay to amplify the oligonucleotide sequence and detect said oligonucleotide sequence, thereby detecting the target protein.

10. The assay of claim 9, wherein the substrate is functionalized with Streptavidin and the first antibody is biotinylated for binding to the Streptavidin.

11. The assay of claim 9, wherein the substrate comprises surfaces of a plurality of magnetic beads.

12. The assay of claim 11, further comprising utilizing, subsequent to the incubation step, a magnetic field to collect the magnetic beads.

13. The assay of claim 12, wherein said incubation step is performed during a time period in a range of about 30 to about 40 minutes.

14. A method for screening an individual for a cancer mediated by HPV infection, comprising:
acquiring a biological specimen from the individual,
analyzing the biological specimen to detect HPV and at least one of a phenotypic biomarker, a regulatory biomarker, a metabolomic biomarker, and a microbiome-derived biomarker dysregulated via HPV injection,
wherein detection of both of the HPV and at least one of said biomarkers indicates the individual is at risk of said cancer.

15. The method of claim 14, wherein said cancer is any of a cervical cancer, a head-and-neck cancer, a penile cancer, a vagina cancer, a valvar cancer and an anal cancer.

16. The method of claim 14, wherein the phenotypic biomarker comprises any of a protein and an mRNA.

17. The method of claim 16, wherein the regulatory biomarker comprises an miRNA.

18. The method of claim 16, wherein the step of analyzing the biological sample to detect the HPV comprises detecting at least one nucleotide target sequences associated with the HPV genome.

19. The method of claim 16, wherein the step of analyzing the biological sample to detect the HPV comprises detecting at least an mRNA associated with transcription of the HPV genome in an infected cell.

20. The method of claim 16, wherein any of said at least one phenotypic biomarker is associated with a cellular molecular process affected by the HPV infection.

21. The method of claim 16, wherein said at least one protein biomarker comprises p-16 protein and EGF receptor.

22. The method of claim 16, further comprising triaging the individual for MRI imaging if the individual is found to be at risk of head and neck cancer.

23-33. (canceled)


SEQUENCE LISTING (24 sequences)

SEQ ID NO: 1 - DNA, 18 bp, synthetic construct
  source 1..18: mol_type = other DNA
tcggttgtgcgtacaaag

SEQ ID NO: 2 - DNA, 21 bp, synthetic construct
  source 1..21: mol_type = other DNA
agcctctacataaaaccatcc

SEQ ID NO: 3 - DNA, 43 bp, synthetic construct
  source 1..43: mol_type = other DNA
tggggcacacaattcctagtttttcacacacgtagacattcgt

SEQ ID NO: 4 - DNA, 48 bp, synthetic construct
  source 1..48: mol_type = other DNA
tcagaaaccataatctaccatggcttttattacatcccgtaccctctt

SEQ ID NO: 5 - DNA, 23 bp, synthetic construct
  source 1..23: mol_type = other DNA
cccattaacaggtcttccaaagt

SEQ ID NO: 6 - DNA, 19 bp, synthetic construct
  source 1..19: mol_type = other DNA
cctgcaggtaccaatgggg

SEQ ID NO: 7 - DNA, 18 bp, synthetic construct
  source 1..18: mol_type = other DNA
aacgacgattccacaaca

SEQ ID NO: 8 - DNA, 20 bp, synthetic construct
  source 1..20: mol_type = other DNA
caaccggaatttcattttgg

SEQ ID NO: 9 - DNA, 43 bp, synthetic construct
  source 1..43: mol_type = other DNA
gtctttcctgtcgtgctcggtttttagctgggcactatagagg

SEQ ID NO: 10 - DNA, 48 bp, synthetic construct
  source 1..48: mol_type = other DNA
cgacgcagagaaacacaagtttttctctaaatgcaatacaatgtcttg

SEQ ID NO: 11 - DNA, 18 bp, synthetic construct
  source 1..18: mol_type = other DNA
cagcacgaatggcactgg

SEQ ID NO: 12 - DNA, 25 bp, synthetic construct
  source 1..25: mol_type = other DNA
tattaagtatgcatggacctaaggc

SEQ ID NO: 13 - DNA, 23 bp, synthetic construct
  source 1..23: mol_type = other DNA
agaagaaaaacaaagacatttgg

SEQ ID NO: 14 - DNA, 18 bp, synthetic construct
  source 1..18: mol_type = other DNA
ctcctcatctgagctgtc

SEQ ID NO: 15 - DNA, 47 bp, synthetic construct
  source 1..47: mol_type = other DNA
gtcttctccaacatgctatgcattttgaaacgattccacaacatagg

SEQ ID NO: 16 - DNA, 47 bp, synthetic construct
  source 1..47: mol_type = other DNA
gagaaacacctacgttgcaagattttgggtaattgctcataacagtg

SEQ ID NO: 17 - DNA, 19 bp, synthetic construct
  source 1..19: mol_type = other DNA
acgtcctgtccaccttcct

SEQ ID NO: 18 - DNA, 25 bp, synthetic construct
  source 1..25: mol_type = other DNA
tgtgttagatttgcaacctgaggca

SEQ ID NO: 19 - DNA, 23 bp, synthetic construct
  source 1..23: mol_type = other DNA
tgcatggagaaataactacattg

SEQ ID NO: 20 - DNA, 19 bp, synthetic construct
  source 1..19: mol_type = other DNA
cgcctcacatttacaacag

SEQ ID NO: 21 - DNA, 54 bp, synthetic construct
  source 1..54: mol_type = other DNA
tgtcacacaattgctcataacagtattttcaagactatgttttagatttggaac

SEQ ID NO: 22 - DNA, 51 bp, synthetic construct
  source 1..51: mol_type = other DNA
gctcagaggaggaggaagatacttttgacgttacaatattataattggagg

SEQ ID NO: 23 - DNA, 22 bp, synthetic construct
  source 1..22: mol_type = other DNA
tattgacggtccagctggacaa

SEQ ID NO: 24 - DNA, 19 bp, synthetic construct
  source 1..19: mol_type = other DNA
ggacaagcaaaaccagaca